SEQUENCE LISTING (34 sequences)

SEQ ID NO: 1 - DNA, 23 bp, Artificial
  An artificially synthesized primer for RT-PCR
catccacgaaactaccttcaact

SEQ ID NO: 2 - DNA, 23 bp, Artificial
  An artificially synthesized primer for RT-PCR
tctccttagagagaagtggggtg

SEQ ID NO: 3 - DNA, 23 bp, Artificial
  An artificially synthesized primer for RT-PCR
ccaattgctatggttacttcagg

SEQ ID NO: 4 - DNA, 23 bp, Artificial
  An artificially synthesized primer for RT-PCR
gaaaaacaagcaggagttgagtg

SEQ ID NO: 5 - DNA, 41 bp, Artificial
  An artificially synthesized primer for RT-PCR
ttaattaactcgagggatcccccttcgaacaaaaactcatc

SEQ ID NO: 6 - DNA, 19 bp, Artificial
  An artificially synthesized primer for RT-PCR
ggcgagaaaggaagggaag

SEQ ID NO: 7 - DNA, 29 bp, Artificial
  An artificially synthesized primer for RT-PCR
atcagatctatggtgagcaagggcgagga

SEQ ID NO: 8 - DNA, 26 bp, Artificial
  An artificially synthesized primer for RT-PCR
atcttacttgtacagctcgtccatgc

SEQ ID NO: 9 - DNA, 27 bp, Artificial
  An artificially synthesized primer for RT-PCR
aatagatatccgcccctctccctcccc

SEQ ID NO: 10 - DNA, 25 bp, Artificial
  An artificially synthesized primer for RT-PCR
aataggatccggcaccgggcttgcg

SEQ ID NO: 11 - DNA, 33 bp, Artificial
  An artificially synthesized primer for PCR
aatattaattaaggaagatggcttccagaagca

SEQ ID NO: 12 - DNA, 30 bp, Artificial
  An artificially synthesized primer for PCR
aataggatcctggtcggtacttgcacagga

SEQ ID NO: 13 - DNA, 1518 bp, Homo sapiens
  CDS (441)..(917)
ataagacttttatggatggattgtttttctcaaataatattatcgcttagtgactaaagtaaagattattaattcctgaggcaagaagatataaaagctccagaaacgttgactgggaccactggagacactgaagaaggcaggggcccttagagtcttggttgccaaacagaatgcccatatccgtcttacctgtgaggaagcttgccttgggcgccctctgctggccctcctgaagctaacaggggcgagtgctcggtggtttacaaattgcctccatgcagactatgaaactgttcagcctgctatagttagatctctggcactggcccaggaggtcttgcagatttgcagatcaaggagaacccaggagtttcaaagaagcgctagtaaggtctctgagatccttgcactagctacatcctcagggtaggaggaagatggcttccagaagcatgcggctgctcctattgctgagctgcctggccaaaacaggagtcctgggtgatatcatcatgagacccagctgtgctcctggatggttttaccacaagtccaattgctatggttacttcaggaagctgaggaactggtctgatgccgagctcgagtgtcagtcttacggaaacggagcccacctggcatctatcctgagtttaaaggaagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctagaagagtgcatctccagcctatgaaacagctgggtctttggccataagaagtaaagatttgaagacagaaggaagaaactcaggagtaagcttctagcccccttcagcttctacacccttctgccctctctccattgcctgcaccccaccccagccactcaactcctgcttgtttttcctttggccatgggaaggtttaccagtagaatccttgctaggttgatgtgggccatacattcctttaataaaccattgtgtacataagaggttgctgtgttccagttcagtaatggtgaatgtggaaaagtgaaataagaccaagaaatacacccag

SEQ ID NO: 14 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 15 - DNA, 351 bp, Mus musculus
  misc_feature: An nucleotide sequence of VH of anti-REG4 mous antibody.
gaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacaggcccatctctactgggatttgcttactggggccaagggactctggtcactgtctctgca

SEQ ID NO: 16 - protein, 117 aa, Mus musculus
  MISC_FEATURE: An amino acid sequence of VH of anti-REG4 mous antibody.
EVQLQQSGTVLARPGASVKMSCKASGYTFTSYWMHWVKQRPGQGLEWIGAIYPGNSDTSYNQKFKGKAKLTAVTSTSTAYMELSSLTNEDSAVYYCTGPSLLGFAYWGQGTLVTVSA

SEQ ID NO: 17 - DNA, 15 bp, Mus musculus
  misc_feature: An nucleotide sequence of VH CDR1 of anti-REG4 mous antibody.
agctactggatgcac

SEQ ID NO: 18 - protein, 5 aa, Mus musculus
  MISC_FEATURE: An amino acid sequence of VH CDR1 of anti-REG4 mous antibody.
SYWMH

SEQ ID NO: 19 - DNA, 14 bp, Mus musculus
  misc_feature: An nucleotide sequence of VH CDR2 of anti-REG4 mous antibody.
gatactagctacaa

SEQ ID NO: 20 - protein, 5 aa, Mus musculus
  MISC_FEATURE: An amino acid sequence of VH CDR2 of anti-REG4 mous antibody.
DTSYN

SEQ ID NO: 21 - DNA, 23 bp, Mus musculus
  misc_feature: An nucleotide sequence of VH CDR3 of anti-REG4 mous antibody.
ccatctctactgggatttgctta

SEQ ID NO: 22 - protein, 8 aa, Mus musculus
  MISC_FEATURE: An amino acid sequence of VH CDR3 of anti-REG4 mous antibody.
PSLLGFAY

SEQ ID NO: 23 - DNA, 312 bp, Mus musculus
  misc_feature: An nucleotide sequence of VL of anti-REG4 mous antibody.
gacattgtgatgacccagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactcctcggacgttcggtggaggcaccaagctg

SEQ ID NO: 24 - protein, 104 aa, Mus musculus
  MISC_FEATURE: An amino acid sequence of VL of anti-REG4 mous antibody.
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPRTFGGGTKL

SEQ ID NO: 25 - DNA, 33 bp, Mus musculus
  misc_feature: An nucleotide sequence of VL CDR1 of anti-REG4 mous antibody.
aaggccagtcaggatgtgagtactgctgtagcc

SEQ ID NO: 26 - protein, 11 aa, Mus musculus
  MISC_FEATURE: An amino acid sequence of VL CDR1 of anti-REG4 mous antibody.
KASQDVSTAVA

SEQ ID NO: 27 - DNA, 21 bp, Mus musculus
  misc_feature: An nucleotide sequence of VL CDR2 of anti-REG4 mous antibody.
tcggcatcctaccggtacact

SEQ ID NO: 28 - protein, 7 aa, Mus musculus
  MISC_FEATURE: An amino acid sequence of VL CDR2 of anti-REG4 mous antibody.
SASYRYT

SEQ ID NO: 29 - DNA, 24 bp, Mus musculus
  misc_feature: An nucleotide sequence of VL CDR3 of anti-REG4 mous antibody.
cagcaacattatagtactcctcgg

SEQ ID NO: 30 - protein, 8 aa, Mus musculus
  MISC_FEATURE: An amino acid sequence of VL CDR3 of anti-REG4 mous antibody.
QQHYSTPR

SEQ ID NO: 31 - DNA, 408 bp, Mus musculus
  misc_feature: An nucleotide sequence of VH of anti-REG4 mous antibody withsugnal sequence.
atggaatgtaactggatacttccttttattctgtcggtaacttcaggggtctactcagaggttcagctccagcagtctgggactgtgctggcaaggcctggggcttcagtgaagatgtcctgcaaggcttctggctacacctttaccagctactggatgcactgggtaaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacaggcccatctctactgggatttgcttactggggccaagggactctggtcactgtctctgca

SEQ ID NO: 32 - protein, 136 aa, Mus musculus
MECNWILPFILSVTSGVYSEVQLQQSGTVLARPGASVKMSCKASGYTFTSYWMHWVKQRPGQGLEWIGAIYPGNSDTSYNQKFKGKAKLTAVTSTSTAYMELSSLTNEDSAVYYCTGPSLLGFAYWGQGTLVTVSA

SEQ ID NO: 33 - DNA, 384 bp, Mus musculus
  misc_feature: An nucleotide sequence of VL of anti-REG4 mous antibody withsugnal sequence.
atgggcatcaaaatggagtcacagattcaggtctttgtattcgtgtttctctggttgtctggtgttgacggagacattgtgatgacccagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactcctcggacgttcggtggaggcaccaagctg

SEQ ID NO: 34 - protein, 128 aa, Mus musculus
MGIKMESQIQVFVFVFLWLSGVDGDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPRTFGGGTKL

(19) United States
(12) Patent Application Publication
(10) Pub. No.: US 2009/0155799 A1
(43) Pub. Date: Jun. 18, 2009

(54) METHODS FOR DIAGNOSING PANCREATIC CANCER USING REG4 PROTEIN

(76) Inventors: Yusuke Nakamura, Bunkyo-ku (JP); Hidewaki Nakagawa, Tokyo (JP); Shuichi Nakatsuru, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/281,206

(22) PCT Filed: Feb. 28, 2007
(86) PCT No.: PCT/JP2007/054375

§ 371 Date: Dec. 10, 2008

Related U.S. Application Data
(60) Provisional application No. 60/779,161, filed on Mar. 2, 2006.

(51) Int. Cl. C12Q 1/68; G01N 33/574; C12Q 1/02; G01N 33/53; C07K 16/00

(52) U.S. Cl. 435/6; 436/64; 435/29; 435/7.92; 435/7.94; 530/389.1; 530/388.1; 436/16

(57) ABSTRACT

REG4, a new member of REG family was identified as a biomarker of pancreatic adenocarcinoma. The present invention provides sandwich ELISA to detect serum REG4 in patients with resectable pancreatic cancers i.e. PDACs. The present invention also provides a method for diagnosing a pancreatic cancer using REG4 as a serological marker.

This application is a U.S. National Phase of PCT/JP2007/054375, filed Feb. 28, 2007, which_claims the benefit of U.S. Provisional Application Ser. No. 60/779,161 filed Mar. 2, 2006, the contents of which are hereby incorporated by reference in their entirety.

### FIELD OF THE INVENTION

The present invention relates to the field of biological science, more specifically to the field of cancer diagnosis. In particular, the present invention relates to methods for diagnosing pancreatic cancer using REG4 protein as a serological marker, and reagents and kits used for the diagnosis.

### BACKGROUND OF THE INVENTION

Pancreatic ductal adenocarcinoma (PDAC) is the fifth leading cause of cancer death in the western world and shows the worst mortality among malignancies, with a 5-year survival rate of only 4% (DiMagno E P, et al., (1999) Gastroenterology.; 117(6): 1464-84., Zervos E E, et al., (2004) Cancer Control.; 11(1): 23-31.). Approximately 30,700 patients are diagnosed with pancreatic cancer in the United States, and nearly 30,000 of them will die of the disease (Jemal A, et al., (2003) CA Cancer J Clin.; 53(1): 5-26.). Since the majority of PDAC patients are diagnosed at an advanced stage, no effective therapy is available at present; only surgical resection offers a little possibility for cure, but 80-90% of PDAC patients who undergo surgery relapse and die from the disease (DiMagno E P, et al, (1999)

Gastroenterology.; 117(6): 1464-84., Zervos E E, et al, (2004) Cancer Control.; 11(11):23-31). Some approaches in surgery and chemotherapy, including 5-FU or gemcitabine, with or without radiation, can improve patients' quality of life (DiMagno E P, et al, (1999) Gastroenterology.; 117(6):1464-84., Zervos E E, et al., (2004) Cancer Control.; 11(1):23-31), but those treatments have a very limited effect on long-term survival of PDAC patients due to its extremely aggressive and chemo-resistant nature. Hence, the management of most patients with advanced PDAC is focused on palliative measures (DiMagno E P, et al., (1999) Gastroenterology.; 117(6):1464-84., Zervos E E, et al., (2004) Cancer Control.; 11(1):23-31).

This horrible prognosis of PDAC arises from several causes, including the difficulty to detect early-staged PDACs (Zervos E E, et al., (2004) Cancer Control.; 11(1):23-31). Despite improvements in diagnostic imaging such as endoscopic ultrasonography (EUS) or magnetic resonance cholangiopancreatography (MRCP), most patients do not develop symptoms until late in the course of their disease and, therefore, do not undergo imaging procedures until their symptom is manifested. An accurate and easy serological test, such as prostate-specific antigen (PSA) in prostate cancer, could facilitate detection of early-staged PDACs without manifested symptom and screening by such a test can be applied to a large-numbered population to detect early-staged PDACs. The surgical resection of early-staged pancreatic cancer can provide the relatively favor prognosis, 50-60% of five-year survival, while the survival rate of advanced pancreatic cancers is only a few % (Zervos E E, et al., (2004) Cancer Control.; 11(1):23-31). Considering the biological aggressiveness and resistance to chemotherapy of PDACs, one of the most realistic strategy to improve the prognosis of fatal PDACs is to screen the high-risk population by non-invasive serological test and to detect early-staged PDACs in which surgical resection can cure. Currently CA19-9 is the only commercially available serological marker for PDACs, however, approximately 10-15% of individuals do not secrete CA19-9 because of their Lewis antigen status, and CA19-9 level is usually within the normal range while pancreatic cancer is at early stage and asymptomatic and it has poor discriminatory value (Sawabu N, et al, (2004) Pancreas.;28(3):263-7., Pleskow D K, et al., (1989) Ann Intern Med.; 110(9):704-9). Hence identification of a novel tumor maker for PDACs and establishment of a screening strategy to detect early-staged pancreatic cancers by using such a serological marker as PSA in prostate cancer are urgently required.

### SUMMARY OF THE INVENTION

The present invention is based on the discovery that the REG4 gene is specifically overexpressed in pancreatic cancer.

Among dozens of genes that were identified as up-regulated in PDAC cells during the present inventors' genome-wide cDNA microarray analysis (Nakamura T, et al., (2004) Oncogene.; 23(13):2385-400, WO2004/031412), the present inventors focused on REG4 (GenBank Accession NO. AY126670; the nucleotide sequence of SEQ ID NO: 13 encoding the amino acid sequence of SEQ ID NO: 14) for this study. The present inventors' microarray data on 20 microdissected-PDAC cell populations had shown a high level of up-regulation of REG4 in 10 of the informative cases examined (Nakamura T, et al., (2004) Oncogene; 23(13):2385-400), and this time its over-expression was confirmed by RT-PCR in six of the twelve microdissected-PDAC cell populations examined as well (FIG. 1A), which had been used for the previous microarray analysis. Although, in the previous studies, REG4 was also referred as REGIV (GenBank Accession Number AA316525), REG4 and REGIV are same molecule.

While the present inventors have identified the REG4 gene as up-regulated in pancreatic cancer tissues, the finding of elevated levels of REG4 in the blood of PDAC patients who would be expected to have early-staged cancer or good prognosis is novel to the present invention. Moreover, the elevated levels of REG4 in the blood of pancreatic-cancer patients suggest that this gene and its protein may be useful as novel diagnostic markers (i.e. whole blood, serum, or plasma). Furthermore, the present inventors established sandwich ELISA to detect serum REG4 in patients with resectable PDACs.

Accordingly, the present invention provides methods for diagnosing pancreatic cancer in a subject comprising the steps of determining the level of REG4 in a subject-derived blood samples and comparing this level to that found in a reference sample, typically a normal control. A high level of REG4 in a sample indicates that the subject either suffers from or is at risk for developing pancreatic cancer. The term “normal control level” indicates a level associated with a normal, healthy individual or a population of individuals known not to be suffering from pancreatic cancer.

The level of REG4 may be determined by detecting the REG4 protein using immunoassay such as ELISA.

Subject-derived blood samples may be derived from whole blood, serum, and plasma derived from subjects, e.g., patients known to or suspected of having pancreatic cancer.

In addition, the present invention provides the above-described methods further comprising the steps of determining the level of CA19-9 in a subject-derived blood samples and comparing the CA19-9 level to that found in a reference sample, typically a normal control. The present inventors found that patients with pancreatic cancer can be identified more sensitively by considering both REG4 and CA19-9 levels.

Furthermore, the present invention also provides immunoassay reagents for detecting REG4 comprising anti-REG4 antibody. The anti-REG4 antibody may comprise polyclonal antibody and monoclonal antibody or at least two monoclonal antibodies recognizing different antigenic determinants of REG4 each other.

Finally, the present invention also provides kits for detecting a pancreatic cancer comprising (i) an immunoassay reagent for determining a level of REG4 in a blood sample; and (ii) a positive control sample for REG4. The kits may further comprise (iii) an immunoassay reagent for determining a level of CA19-9 in a blood sample; and (iv) a positive control sample for CA19-9.

Other features and advantages of the invention will be apparent from the following detailed description and from the claims. It is to be understood that both the foregoing summary of the present invention and the following detailed descriptions are of a preferred embodiment, and not restrictive of the invention or other alternate embodiments of the present invention.

### BRIEF DESCRIPTION OF THE DRAWINGS

FIG. 1 shows: (A) RT-PCR for the mRNA expression of REG4 and ACTB in the microdissected PDAC cells (1-12) comparing with normal pancreatic ductal epithelial cells (N) which were also microdissected; and (B), in immunohistochemical study using anti-REG4 antibody, intense staining in some PDAC cells (upper panel, original magnification ×200). Positive staining of REG4 was observed at the cytoplasm. In normal pancreatic tissue, acinar cells showed weak staining (lower panel, original magnification ×200), but not in normal ductal epithelium cells and islet cells.

FIG. 2 shows the standard curve of the sandwich ELISA for determination of the REG4 level.

FIG. 3 shows the distribution of levels of REG4 in 123 healthy people. Serum REG4 concentrations were determined by ELISA method.

FIG. 4 shows serum REG4 level at the pre-operation and post-operation of seven patients with resectable PDACs. Open bar, pre-operation; and closed bar, post-operation. Normal range of serum REG4 was putatively defined below 9.0 ng/mL (dotted line).

FIG. 5 shows a standard curve of the modified sandwich ELISA for determination of the REG4 level.

FIG. 6 shows: (A) the list of diagnostic result using REG4 or CA19-9 as marker; and (B) the Venn diagram of overlap between REG4-positive population and CA19-9-positive population. “+” indicates positive result; and “−” indicates negative result. There were 59 pancreatic cancer samples, 29 REG4-positive samples and 45 CA19-9-positive samples. Among them, 22 samples were positive for both REG4 and CA19-9 and 7 samples were negative for both REG4 and CA19-9.

FIG. 7 shows a standard curve of the sandwich ELISA using each anti-REG4 monoclonal antibody (clone 21-1, 24-1, 34-1) for determination of the REG4 level.

FIG. 8 depicts the detection of REG4 protein in serum of 9 pancreatic cancer patients and 28 healthy people.

### DETAILED DESCRIPTION OF THE INVENTION

The terms “a”, “an”, and “the” as used herein mean “at least one” unless otherwise specifically indicated.

In the present invention, REG4, a new member of REG (regenerating islet-derived) family (Hartupee J C, et al., (2001) Biochim Biophys Acta.; 1518(3):287-93) was identified as a biomarker of pancreatic adenocarcinoma. The present inventors have reported genome-wide cDNA microarrays of microdissected pancreatic cancer cells (Nakamura T, et al., (2004) Oncogene.; 23(13):2385-400). Although, in this report, REG4 was found to be over-expressed in pancreatic cancer cells, it provides no evidence as to how the level of REG4 in blood relates to pancreatic cancer. REG family are secreted molecules associated with tissue regeneration and inflammation in digestive organs (Hartupee J C, et al., (2001) Biochim Biophys Acta.; 1518(3):287-93., Watanabe T, et al., (1990) J Biol. Chem.; 265(13):7432-9., Unno M, et al., (1992) Adv Exp Med. Biol.; 321:61-6; discussion 67-9) and REG family are up-regulated in several gastrointestinal cancers (Unno M, et al., (1992) Adv Exp Med. Biol.; 321:61-6; discussion 67-9., Lasserre C, et al., (1992) Cancer Res.; 52(18):5089-95., Kamarainen M, et al, (2003) Am J. Pathol.; 163(1):11-20) and may function as a trophic or anti-apoptotic factor in cancers (Lasserre C, et al., (1992) Cancer Res.; 52(18):5089-95), but their pathophysiological functions, especially the function and expression pattern of a novel member, REG4, are still unclear. The present inventors have generated REG4-specific antibodies and validated its overexpression of PDAC cells by immunohistochemistry using anti-REG4 antibody. Furthermore, the present inventors have established sandwich ELISA systems to measure REG4 level in serum of patients with PDACs, and demonstrated the use of REG4 as a new serological marker of pancreatic cancer.

### Diagnosing Pancreatic Cancer:

By measuring the level of REG4 in subject-derived blood samples, the occurrence of pancreatic cancer or a predisposition to develop pancreatic cancer in a subject can be determined. Accordingly, the present invention involves determining (e.g., measuring) the level of REG4 in blood samples. In the present invention, a method for diagnosing pancreatic cancer also includes a method for testing or detecting pancreatic cancer. Alternatively, in the present invention, diagnosing pancreatic cancer also refers to showing a suspicion, risk, or possibility of pancreatic cancer in a subject.

Any blood samples may be used for determining the level of REG4 so long as either the REG4 gene or the REG4 protein can be detected in the samples. Preferably, the blood samples comprise whole blood, serum, and plasma.

In the present invention, the “level of REG4 in blood samples” refers to the concentration of REG4 present in the blood after correcting the corpuscular volume in the whole blood. One of skill will recognize that the percentage of corpuscular volume in the blood varies greatly between individuals. For example, the percentage of erythrocytes in the whole blood is very different between men and women. Furthermore, differences between individuals cannot be ignored. Therefore, the apparent concentration of a substance in the whole blood which comprises corpuscular components varies greatly depending on the percentage of corpuscular volume. For example, even if the concentration in the serum is the same, the measured value for a sample with a large amount of corpuscular component will be lower than the value for a sample with a small amount of corpuscular component. Therefore, to compare the measured values of components in the blood, values for which the corpuscular volume has been corrected are usually used.

For example, by measuring components in the blood using, as samples, serum or plasma obtained by separating blood cells from the whole blood, measured values from which the effect from the corpuscular volume has been removed can be obtained. Therefore, the level of REG4 in the present invention can usually be determined as a concentration in the serum or plasma. Alternatively, it may first be measured as a concentration in the whole blood, then the effect from the corpuscular volume may be corrected. Methods for measuring a corpuscular volume in a whole blood sample are known.

Subjects diagnosed for pancreatic cancer according to the present methods are preferably mammals and include humans, non-human primates, mice, rats, dogs, cats, horses and cows. A preferable subject of the present invention is a human. In the present invention, a subject may be a patient suspected of having cancer of the gastrointestinal system (e.g. pancreas) or healthy individuals. The patient may be diagnosed by the present invention to facilitate clinical decision-making. In another embodiment, the present invention may also be applied to healthy individuals for screening of cancer of gastrointestinal system (i.e. pancreas).

In one embodiment of the present invention, the level of REG4 is determined by measuring the quantity or concentration of REG4 protein in blood samples. Methods for determining the quantity of the REG4 protein in blood samples include immunoassay methods.

In the methods of diagnosis of the present invention, the blood concentration of CA19-9 may be determined, in addition to the blood concentration of REG4, to detect pancreatic cancer. Therefore, the present invention provides methods for diagnosing pancreatic cancer, in which pancreatic cancer is detected when either the blood concentration of REG4 or the blood concentration of CA19-9, or both of them, are higher as compared with healthy individuals.

It is well known that CA19-9 is a serological tumor marker for pancreatic, colon, gastric and ovarian carcinomas. The epitope of CA19-9 is a glycolipid on a glycoprotein (mucin) which corresponds to the Lewis (a) blood group determinant with added sialic acid residues. The antigen is defined by a monoclonal antibody raised against determinants found in human colorectal cancer cell lines. The antigen is also found in normal fetal tissue as well as adult pancreas, salivary ducts, gastric and colonic epithelium, pancreatic fluid, gastric fluid, saliva and meconium. CA 19-9 is removed from the circulation by the biliary system. The antigen is not expressed in persons with genotype Lewis (a-b-), which corresponds to about 5% of the population.

As described above, CA19-9 has already been used as serological marker for diagnosing or detecting pancreatic cancer. However, the sensitivity of CA19-9 as a marker for pancreatic cancer is somewhat insufficient for detecting pancreatic cancer, completely. Accordingly, it is required that the sensitivity of diagnosing pancreatic cancer would be improved.

In the present invention, a novel serological marker for pancreatic cancer, REG4, is provided. Improvement in the sensitivity of diagnostic or detection methods for pancreatic cancer may be achieved by the present invention. Namely, the present invention provides a method for diagnosing pancreatic cancer in a subject, comprising the steps of:
(a) collecting a blood sample from a subject to be diagnosed;
(b) determining a level of REG4 in the blood sample;
(c) comparing the REG4 level determined in step (b) with that of a normal control wherein a high REG4 level in the blood sample, compared to the normal control, indicates that the subject suffers from pancreatic cancer.

In preferable embodiments, the diagnostic or detection method of the present invention may further comprise the steps of:
(d) determining a level of CA19-9 in the blood sample;
(e) comparing the CA19-9 level determined in step (e) with that of a normal control; and
(f) judging that either or both of high REG4 and high CA19-9 levels in the blood sample, compared to the normal control, indicate that the subject suffers from pancreatic cancer.

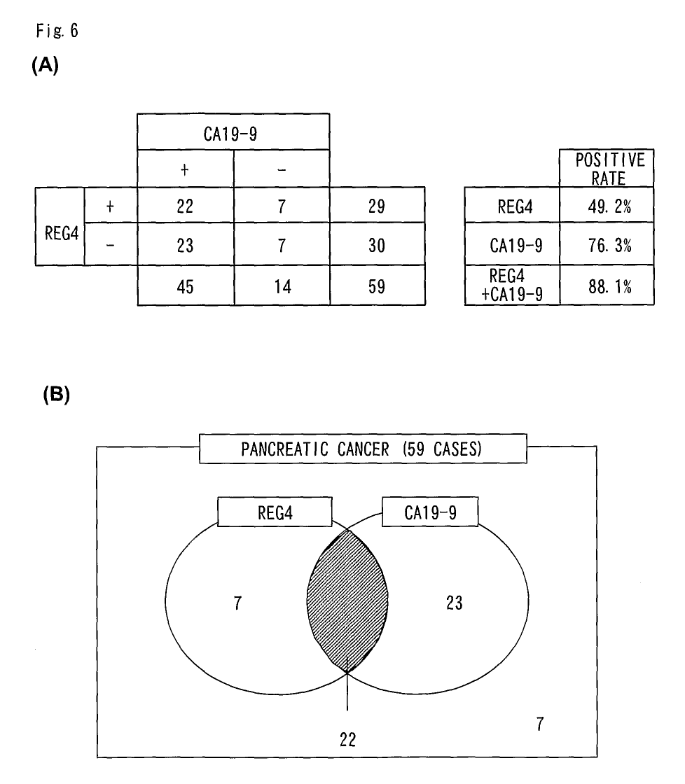

By the combination between REG4 and CA19-9, the sensitivity for detection of pancreatic cancer may be significantly improved. For example, in the group analyzed in the working example discussed below, positive rate of CA19-9 for pancreatic cancer is about 76.3%. In comparison, that of combination between CA19-9 and REG4 increases to 88.1% (FIG. 6). In the present invention, “combination of CA19-9 and REG4” refers to either or both levels of CA19-9 and REG4 being used as marker. In the preferable embodiments, a patient with positive either of CA19-9 and REG4 may be judged to have a high risk of pancreatic cancer. The use of combination of REG4 and CA19-9 as serological marker for pancreatic cancer is novel.

Therefore, the present invention can greatly improve the sensitivity for detecting pancreatic cancer patients, compared to determinations based on results of measuring CA19-9 alone. Behind this improvement is the fact that the group of CA19-9-positive patients and the group of REG4-positive patients do not match completely. This fact is further described specifically below.

First, among patients who, as a result of CA19-9 measurements, were determined to have a lower value than a standard value (i.e. not to have pancreatic cancer), there is actually a certain percentage of patients that have pancreatic cancer. Such patients are referred to as CA19-9-false negative patients. By combining a determination based on CA19-9 with a determination based on REG4, patients whose REG4 value is above the standard value can be found from among the CA19-9-false-negative patients. That is, from among patients falsely determined to be “negative” due to a low blood concentration of CA19-9, the present invention provides a means to identify patients actually having pancreatic cancer. The sensitivity for detecting pancreatic cancer patients is thus improved by the present invention. Generally, simply combining the results from determinations using multiple markers may increase the detection sensitivity, but on the other hand, it often causes a decrease in specificity. However, by determining the best balance between sensitivity and specificity, the present invention has determined a characteristic combination that can increase the detection sensitivity without compromising the specificity.

In the present invention, in order to consider the results of CA19-9 measurements at the same time, for example, the blood concentration of CA19-9 may be measured and compared with standard values, in the same way as for the aforementioned comparison between the measured values and standard values of REG4. For example, how to measure the blood concentration of CA19-9 and compare it to standard values are already known. Moreover, ELISA kits for CA19-9 are also commercially available. These methods described in known reports can be used in the method of the present invention for diagnosing or detecting pancreatic cancer.

In the present invention, the standard value of the blood concentration of REG4 can be determined statistically. For example, the blood concentration of REG4 in healthy individuals can be measured to determine the standard blood concentration of REG4 statistically. When a statistically sufficient population is gathered, a value in the range of twice or three times the standard deviation (S.D.) from the mean value is often used as the standard value. Therefore, values corresponding to the mean value+2×S.D. or mean value+3×S.D. may be used as standard values. The standard values set as described theoretically comprise 90% and 99.7% of healthy individuals, respectively.

Alternatively, standard values can also be set based on the actual blood concentration of REG4 in pancreatic cancer patients. Generally, standard values set this way minimize the percentage of false positives, and are selected from a range of values satisfying conditions that can maximize detection sensitivity. Herein, the percentage of false positives refers to a percentage, among healthy individuals, of patients whose blood concentration of REG4 is judged to be higher than a standard value. On the contrary, the percentage, among healthy individuals, of patients whose blood concentration of REG4 is judged to be lower than a standard value indicates specificity. That is, the sum of the false positive percentage and the specificity is always 1. The detection sensitivity refers to the percentage of patients whose blood concentration of REG4 is judged to be higher than a standard value, among all pancreatic cancer patients within a population of individuals for whom the presence of pancreatic cancer has been determined.

Furthermore, in the present invention, the percentage of pancreatic cancer patients among patients whose REG4 concentration was judged to be higher than a standard value represents the positive predictive value. On the other hand, the percentage of healthy individuals among patients whose REG4 concentration was judged to be lower than a standard value represents the negative predictive value. The relationship between these values is summarized in Table 1. As the relationship shown below indicates, each of the values for sensitivity, specificity, positive predictive value, and negative predictive value, which are indexes for evaluating the diagnostic accuracy for pancreatic cancer, varies depending on the standard value for judging the level of the blood concentration of REG4.

TABLE 1

| Blood | Pancreatic | | |
|---|---|---|---|
| concentration | cancer | Healthy | |
| of REG4 | patients | individuals | |
| High | a: True positive | b: False positive | Positive predictive |
| | | | value a/(a + b) |
| Low | c: False | d: True negative | Negative predictive |
| | negative | | value d/(c + d) |
| | Sensitivity | Specificity | |
| | a/(a + c) | d/(b + d) | |

As already mentioned, a standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a “low false positive ratio”. Considering this situation, for example, values that give the following predicted results may be selected as the preferable standard values in the present invention.

Standard values for which the false positive ratio is 50% or less (that is, standard values for which the specificity is not less than 50%).

Standard values for which the sensitivity is not less than 20%.

In the present invention, the standard values can be set using a receiver operating characteristic (ROC) curve. A ROC curve is a graph that shows the detection sensitivity on the vertical axis and the false positive ratio (that is, “1-specificity”) on the horizontal axis. In the present invention, an ROC curve can be obtained by plotting the changes in the sensitivity and the false positive ratio, which were obtained after continuously varying the standard value for determining the high/low degree of the blood concentration of REG4.

The “standard value” for obtaining the ROC curve is a value temporarily used for the statistical analyses. The “standard value” for obtaining the ROC curve can generally be continuously varied within a range that allows to cover all selectable standard values. For example, the standard value can be varied between the smallest and largest measured REG4 values in an analyzed population.

Based on the obtained ROC curve, a preferable standard value to be used in the present invention can be selected from a range that satisfies the above-mentioned conditions. Alternatively, a standard value can be selected based on an ROC curve produced by varying the standard values from a range that comprises most of the measured REG4 values.

REG4 in the blood can be measured by any method that can quantitate proteins. For example, immunoassay, liquid chromatography, surface plasmon resonance (SPR), mass spectrometry, or the like can be used in the present invention. In mass spectrometry, proteins can be quantitated by using a suitable internal standard. For example, isotope-labeled REG4 can be used as the internal standard. The concentration of REG4 in the blood can be determined from the peak intensity of REG4 in the blood and that of the internal standard. Generally, the matrix-assisted laser desorption/ionization (MALDI) method is used for mass spectrometry of proteins. With an analysis method that uses mass spectrometry or liquid chromatography, REG4 can also be analyzed simultaneously with other tumor markers (e.g. CA19-9).

A preferable method for measuring REG4 in the present invention is the immunoassay. The amino acid sequence of REG4 is known (Genbank Accession Number AY126670). The amino acid sequence of REG4 is shown in SEQ ID NO: 14, and the nucleotide sequence of the cDNA encoding it is shown in SEQ ID NO: 13. Therefore, those skilled in the art can prepare antibodies by synthesizing necessary immunogens based on the amino acid sequence of REG4. The peptide used as immunogen can be easily synthesized using a peptide synthesizer. The synthetic peptide can be used as an immunogen by linking it to a carrier protein.

Keyhole limpet hemocyanin, myoglobin, albumin, and the like can be used as the carrier protein. Preferrable carrier proteins are KLH, bovine serum albumin, and such. The maleimidobenzoyl-N-hydrosuccinimide ester method (hereinafter abbreviated as the MBS method) and the like are generally used to link synthetic peptides to carrier proteins.

Specifically, a cysteine is introduced into the synthetic peptide and the peptide is crosslinked to KLH by MBS using the cysteine's SH group. The cysteine residue may be introduced at the N-terminus or C-terminus of the synthesized peptide.

Alternatively, REG4 can be prepared using the nucleotide sequence of REG4 (Genbank Accession Number AY126670), or a portion thereof. DNAs comprising the necessary nucleotide sequence can be cloned using mRNAs prepared from REG4-expressing tissues. Alternatively, commercially available cDNA libraries can be used as the cloning source. The obtained genetic recombinants of REG4, or fragments thereof, can also be used as the immunogen. REG4 recombinants expressed in this manner are preferrable as the immunogen for obtaining the antibodies used in the present invention. Commercially available REG4 recombinants (ProSpec-Tany TechnoGene Ltd., Product No: PRO-424) can also be used as the immunogen.

Immunogens obtained in this manner are mixed with a suitable adjuvant and used to immunize animals. Known adjuvants include Freund's complete adjuvant (FCA) and incomplete adjuvant. The immunization procedure is repeated at appropriate intervals until an increase in the antibody titer is confirmed. There are no particular limitations on the immunized animals in the present invention. Specifically, animals commonly used for immunization such as mice, rats, or rabbits can be used.

When obtaining the antibodies as monoclonal antibodies, animals that are advantageous for their production may be used. For example in mice, many myeloma cell lines for cell fusion are known, and techniques for establishing hybridomas with a high probability are already well known. Therefore, mice are a desirable immunized animal to obtain monoclonal antibodies.

Furthermore, the immunization treatments are not limited to in vitro treatments. Methods for immunologically sensitizing cultured immunocompetent cells in vitro can also be employed. Antibody-producing cells obtained by these methods are transformed and cloned. Methods for transforming antibody-producing cells to obtain monoclonal antibodies are not limited to cell fusion. For example, methods for obtaining clonable transformants by virus infection are known.

Hybridomas that produce the monoclonal antibodies used in the present invention can be screened based on their reactivity to REG4. Specifically, antibody-producing cells are first selected by using as an index the binding activity toward REG4, or a domain peptide thereof, that was used as the immunogen. Positive clones that are selected by this screening are subcloned as necessary.

The monoclonal antibodies to be used in the present invention can be obtained by culturing the established hybridomas under suitable conditions and collecting the produced antibodies. When the hybridomas are homohybridomas, they can be cultured in vivo by inoculating them intraperitoneally in syngeneic animals. In this case, monoclonal antibodies are collected as ascites fluid. When heterohybridomas are used, they can be cultured in vivo using nude mice as a host.

In addition to in vivo cultures, hybridomas are also commonly cultured ex vivo, in a suitable culture environment. For example, basal media such as RPMI 1640 and DMEM are generally used as the medium for hybridomas. Additives such as animal sera can be added to these media to maintain the antibody-producing ability to a high level. When hybridomas are cultured ex vivo, the monoclonal antibodies can be collected as a culture supernatant. Culture supernatants can be collected by separating from cells after culturing, or by continuously collecting while culturing using a culture apparatus that uses a hollow fiber.

Monoclonal antibodies used in the present invention are prepared from monoclonal antibodies collected as ascites fluid or culture supernatants, by separating immunoglobulin fractions by saturated ammonium sulfate precipitation and further purifying by gel filtration, ion exchange chromatography, or such. In addition, if the monoclonal antibodies are IgGs, purification methods based on affinity chromatography with a protein A or protein G column are effective.

An example of monoclonal antibody that can be used in the immunoassays of the present invention is the mouse monoclonal clone 21-1 antibody. More specifically, mouse monoclonal clone 21-1 antibody, or antibody fragments comprising variable regions thereof, are preferable as monoclonal antibodies used in the immunoassays of the present invention. For example, monoclonal clone 21-1 antibody, or monoclonal antibodies having an equivalent antigen-binding activity as this antibody, are useful as immobilized antibodies for immunoassays that are based on the sandwich method. In a preferred embodiment of the present invention, REG4 can be measured by a sandwich method that uses monoclonal antibodies immobilized onto a carrier and labeled polyclonal antibodies. Such a combination of antibodies is a preferable combination that allows a highly sensitive detection of REG4.

The amino acid sequences of VH and VL of the monoclonal clone 21-1 antibody are shown in SEQ ID NOs: 16 and 24, respectively. One skilled in the art can produce monoclonal antibodies having a same binding activity by genetic engineering based on this amino acid sequence information. Furthermore, by transplanting the CDR1, CDR2, and CDR3 of VH and VL into the framework of other immunoglobulins, antibodies having an equivalent antigen-binding activity can be reconstituted. The CDRs of VH and VL of clone 21-1 are composed of the amino acid sequences shown below. Each amino acid sequence is encoded by the nucleotide sequences of the SEQ ID NOs indicated below. Therefore, by substituting the corresponding CDRs of other immunoglobulins with the DNAs comprising these nucleotide sequences, the antigen-binding activity of clone 21-1 can be transplanted to other immunoglobulins.

| | Nucleotide sequence | Amino acid sequence |
|---|---|---|
| Heavy chain | | |
| CDR1 | SEQ ID NO: 17 | SEQ ID NO: 18 |
| CDR2 | SEQ ID NO: 19 | SEQ ID NO: 20 |
| CDR3 | SEQ ID NO: 21 | SEQ ID NO: 22 |
| Light chain | | |
| CDR1 | SEQ ID NO: 25 | SEQ ID NO: 26 |
| CDR2 | SEQ ID NO: 27 | SEQ ID NO: 28 |
| CDR3 | SEQ ID NO: 29 | SEQ ID NO: 30 |

The clone 21-1 provided by the present invention is a novel monoclonal antibody which is useful for immunoassays of REG4. Thus, the present invention provides anti-REG4 monoclonal antibodies comprising the aforementioned amino acid sequences as the CDRs. The monoclonal antibodies of the present invention preferably comprise the amino acid sequences of SEQ ID NOs: 16 and 24 as the amino acid sequences of VH and VL, respectively.

Immunoglobulins comprising the amino acid sequences of SEQ ID NOs: 16 and 24 in their variable regions can be expressed by incorporating DNAs encoding the amino acid sequences in a suitable expression vector. By expressing the DNAs together with DNAs encoding a constant region, immunoglobulins equipped with a constant region can also be obtained. In the immunoassays of the present invention, complete immunoglobulins equipped with a constant region may be used as the antibody, or immunoglobulin fragments carrying an antigen binding region may also be used as the antibody. A signal sequence can be added to the N-terminus of the variable regions to secrete the expression products from the host cells. Amino acid sequences of VH and VL onto which a signal sequence has been added are shown in SEQ ID NOs: 32 and 34, respectively, and nucleotide sequences encoding these amino acid sequences are shown in SEQ ID NOs: 31 and 33, respectively.

On the other hand, to obtain antibodies used in the present invention as polyclonal antibodies, blood is drawn from animals whose antibody titer increased after immunization, and the serum is separated to obtain an anti-serum. Immunoglobulins are purified from anti-sera by known methods to prepare the antibodies used in the present invention. REG4-specific antibodies can be prepared by combining immunoaffinity chromatography which uses REG4 as a ligand with immunoglobulin purification.

When antibodies against REG4 contact REG4, the antibodies bind to the antigenic determinant (epitope) that the antibodies recognize through an antigen-antibody reaction. The binding of antibodies to antigens can be detected by various immunoassay principles. Immunoassays can be broadly categorized into heterogeneous analysis methods and homogeneous analysis methods. To maintain the sensitivity and specificity of immunoassays to a high level, the use of monoclonal antibodies is desirable. Methods of the present invention for measuring REG4 by various immunoassay formats are specifically explained.

First, methods for measuring REG4 using a heterogeneous immunoassay are described. In heterogeneous immunoassays, a mechanism for detecting antibodies that bound to REG4 after separating them from those that did not bind to REG4 is required.

To facilitate the separation, immobilized reagents are generally used. For example, a solid phase onto which antibodies recognizing REG4 have been immobilized is first prepared (immobilized antibodies). REG4 is made to bind to these, and secondary antibodies are further reacted thereto.

When the solid phase is separated from the liquid phase and further washed, as necessary, secondary antibodies remain on the solid phase in proportion to the concentration of REG4. By labeling the secondary antibodies, REG4 can be quantitated by measuring the signal derived from the label.

Any method may be used to bind the antibodies to the solid phase. For example, antibodies can be physically adsorbed to hydrophobic materials such as polystyrene. Alternatively, antibodies can be chemically bound to a variety of materials having functional groups on their surfaces. Furthermore, antibodies labeled with a binding ligand can be bound to a solid phase by trapping them using a binding partner of the ligand. Combinations of a binding ligand and its binding partner include avidin-biotin and such. The solid phase and antibodies can be conjugated at the same time or before the reaction between the primary antibodies and REG4.

Similarly, the secondary antibodies do not need to be directly labeled. That is, they can be indirectly labeled using antibodies against antibodies or using binding reactions such as that of avidin-biotin.

The concentration of REG4 in a sample is determined based on the signal intensities obtained using standard samples with known REG4 concentrations.

Any antibody can be used as the immobilized antibody and secondary antibody for the heterogeneous immunoassays mentioned above, so long as it is an antibody, or a fragment comprising an antigen-binding site thereof, that recognizes REG4. Therefore, it may be a monoclonal antibody, a polyclonal antibody, or a mixture or combination of both. For example, a combination of monoclonal antibodies and polyclonal antibodies is a preferable combination in the present invention. Alternatively, when both antibodies are monoclonal antibodies, combining monoclonal antibodies recognizing different epitopes is preferable.

Since the antigens to be measured are sandwiched by antibodies, such heterogenous immunoassays are called sandwich methods. Since sandwich methods excel in the measurement sensitivity and the reproducibility, they are a preferable measurement principle in the present invention.

The principle of competitive inhibition reactions can also be applied to the heterogeneous immunoassays. Specifically, they are immunoassays based on the phenomenon where REG4 in a sample competitively inhibits the binding between REG4 with a known concentration and an antibody. The concentration of REG4 in the sample can be determined by labeling REG4 with a known concentration and measuring the amount of REG4 that reacted (or did not react) with the antibody.

A competitive reaction system is established when antigens with a known concentration and antigens in a sample are simultaneously reacted to an antibody. Furthermore, analyses by an inhibitory reaction system are possible when antibodies are reacted with antigens in a sample, and antigens with a known concentration are reacted thereafter. In both types of reaction systems, reaction systems that excel in the operability can be constructed by setting either one of the antigens with a known concentration used as a reagent component or the antibody as the labeled component, and the other one as the immobilized reagent.

Radioisotopes, fluorescent substances, luminescent substances, substances having an enzymatic activity, macroscopically observable substances, magnetically observable substances, and such are used in these heterogeneous immunoassays. Specific examples of these labeling substances are shown below.
Substances having an enzymatic activity:
   peroxidase,
   alkaline phosphatase,
   urease, catalase,
   glucose oxidase,
   lactate dehydrogenase, or
   amylase, etc.
Fluorescent substances:
   fluorescein isothiocyanate,
   tetramethylrhodamine isothiocyanate,
   substituted rhodamine isothiocyanate, or
   dichlorotriazine isothiocyanate, etc.
Radioisotopes:
   tritium,
   ¹²⁵I, or
   ¹³¹I, etc.

Among these, non-radioactive labels such as enzymes are an advantageous label in terms of safety, operability, sensitivity, and such. Enzymatic labels can be linked to antibodies or to REG4 by known methods such as the periodic acid method or maleimide method.

As the solid phase, beads, inner walls of a container, fine particles, porous carriers, magnetic particles, or such are used. Solid phases formed using materials such as polystyrene, polycarbonate, polyvinyltoluene, polypropylene, polyethylene, polyvinyl chloride, nylon, polymethacrylate, latex, gelatin, agarose, glass, metal, ceramic, or such can be used. Solid materials in which functional groups to chemically bind antibodies and such have been introduced onto the surface of the above solid materials are also known. Known binding methods, including chemical binding such as poly-L-lysine or glutaraldehyde treatment and physical adsorption, can be applied for solid phases and antibodies (or antigens).

Although the steps of separating the solid phase from the liquid phase and the washing steps are required in all heterogeneous immunoassays exemplified herein, these steps can easily be performed using the immunochromatography method, which is a variation of the sandwich method.

Specifically, antibodies to be immobilized are immobilized onto porous carriers capable of transporting a sample solution by the capillary phenomenon, then a mixture of a sample comprising REG4 and labeled antibodies is deployed therein by this capillary phenomenon. During deployment, REG4 reacts with the labeled antibodies, and when it further contacts the immobilized antibodies, it is trapped at that location. The labeled antibodies that do not react with REG4 pass through, without being trapped by the immobilized antibodies.

As a result, the presence of REG4 can be detected using, as an index, the signals of the labeled antibodies that remain at the location of the immobilized antibodies. If the labeled antibodies are maintained upstream in the porous carrier in advance, all reactions can be initiated and completed by just dripping in the sample solutions, and an extremely simple reaction system can be constructed. In the immunochromatography method, labeled components that can be distinguished macroscopically, such as colored particles, can be combined to construct an analytical device that does not even require a special reader.

Furthermore, in the immunochromatography method, the detection sensitivity for REG4 can be adjusted. For example, by adjusting the detection sensitivity near the cutoff value described below, the aforementioned labeled components can be detected when the cutoff value is exceeded. By using such a device, whether a subject is positive or negative can be judged very simply. By adopting a constitution that allows a macroscopic distinction of the labels, necessary examination results can be obtained by simply applying blood samples to the device for immunochromatography.

Various methods for adjusting the detection sensitivity of the immunochromatography method are known. For example, a second immobilized antibody for adjusting the detection sensitivity can be placed between the position where samples are applied and the immobilized antibodies (Japanese Patent Application Kokai Publication No. (JP-A) H06-341989 (unexamined, published Japanese patent application)). REG4 in the sample is trapped by the second immobilized antibody while deploying from the position where the sample was applied to the position of the first immobilized antibody for label detection. After the second immobilized antibody is saturated, REG4 can reach the position of the first immobilized antibody located downstream. As a result, when the concentration of REG4 comprised in the sample exceeds a predetermined concentration, REG4 bound to the labeled antibody is detected at the position of the first immobilized antibody.

Next, homogeneous immunoassays are explained. As opposed to heterogeneous immunological assay methods that require a separation of the reaction solutions as described above, REG4 can also be measured using homogeneous analysis methods. Homogeneous analysis methods allow the detection of antigen-antibody reaction products without their separation from the reaction solutions.

A representative homogeneous analysis method is the immunoprecipitation reaction, in which antigenic substances are quantitatively analyzed by examining precipitates produced following an antigen-antibody reaction. Polyclonal antibodies are generally used for the immunoprecipitation reactions. When monoclonal antibodies are applied, multiple types of monoclonal antibodies that bind to different epitopes of REG4 are preferably used. The products of precipitation reactions that follow the immunological reactions can be macroscopically observed or can be optically measured for conversion into numerical data.

The immunological particle agglutination reaction, which uses as an index the agglutination by antigens of antibody-sensitized fine particles, is a common homogeneous analysis method. As in the aforementioned immunoprecipitation reaction, polyclonal antibodies or a combination of multiple types of monoclonal antibodies can be used in this method as well. Fine particles can be sensitized with antibodies through sensitization with a mixture of antibodies, or they can be prepared by mixing particles sensitized separately with each antibody. Fine particles obtained in this manner gives matrix-like reaction products upon contact with REG4. The reaction products can be detected as particle aggregation. Particle aggregation may be macroscopically observed or can be optically measured for conversion into numerical data.

Immunological analysis methods based on energy transfer and enzyme channeling are known as homogeneous immunoassays. In methods utilizing energy transfer, different optical labels having a donor/acceptor relationship are linked to multiple antibodies that recognize adjacent epitopes on an antigen. When an immunological reaction takes place, the two parts approach and an energy transfer phenomenon occurs, resulting in a signal such as quenching or a change in the fluorescence wavelength. On the other hand, enzyme channeling utilizes labels for multiple antibodies that bind to adjacent epitopes, in which the labels are a combination of enzymes having a relationship such that the reaction product of one enzyme is the substrate of another. When the two parts approach due to an immunological reaction, the enzyme reactions are promoted; therefore, their binding can be detected as a change in the enzyme reaction rate.

In the present invention, blood for measuring REG4 can be prepared from blood drawn from patients. Preferable blood samples are the serum or plasma. Serum or plasma samples can be diluted before the measurements. Alternatively, the whole blood can be measured as a sample and the obtained measured value can be corrected to determine the serum concentration. For example, concentration in whole blood can be corrected to the serum concentration by determining the percentage of corpuscular volume in the same blood sample.

In a preferred embodiment, the immunoassay comprises an ELISA. The present inventors established sandwich ELISA to detect serum REG4 in patients with resectable PDACs.

The REG4 level in the blood samples is then compared with an REG4 level associated with a reference sample such as a normal control sample. The phrase “normal control level” refers to the level of REG4 typically found in a blood sample of a population not suffering from pancreatic cancer. The reference sample is preferably of a similar nature to that of the test sample. For example, if the test samples comprise patient serum, the reference sample should also be serum. The REG4 level in the blood samples from control and test subjects may be determined at the same time or, alternatively, the normal control level may be determined by a statistical method based on the results obtained by analyzing the level of REG4 in samples previously collected from a control group.

The REG4 level may also be used to monitor the course of treatment of pancreatic cancer. In this method, a test blood sample is provided from a subject undergoing treatment for pancreatic cancer. Preferably, multiple test blood samples are obtained from the subject at various time points before, during, or after the treatment. The level of REG4 in the post-treatment sample may then be compared with the level of REG4 in the pre-treatment sample or, alternatively, with a reference sample (e.g., a normal control level). For example, if the post-treatment REG4 level is lower than the pre-treatment REG4 level, one can conclude that the treatment was efficacious. Likewise, if the post-treatment REG4 level is similar to the normal control REG4 level, one can also conclude that the treatment was efficacious.

An “efficacious” treatment is one that leads to a reduction in the level of REG4 or a decrease in size, prevalence, or metastatic potential of pancreatic cancer in a subject. When a treatment is applied prophylactically, “efficacious” means that the treatment retards or prevents occurrence of pancreatic cancer or alleviates a clinical symptom of pancreatic cancer. The assessment of pancreatic cancer can be made using standard clinical protocols. Furthermore, the efficaciousness of a treatment can be determined in association with any known method for diagnosing or treating pancreatic cancer. For example, pancreatic cancer is routinely diagnosed histopathologically or by identifying symptomatic anomalies.

According to the results from the Examples described below, REG4, which is a serological marker provided by the present invention for pancreatic cancer, may show a high measured value in patients having a cancer other than pancreatic cancer as well. Specifically, a high blood concentration was observed particularly for stomach cancer and colon cancer. A high REG4 expression was actually confirmed by immunohistological staining in stomach cancer (Oue N., et al., (2005) J. Pathol., 207(2):185-98) and colon cancer (Violette S., et al., (2003) Int. J. Cancer, 103(2):185-93). However, the possibility of having such cancers can be easily ruled out by using other diagnostic indicators. Therefore, the possibility that a patient judged to have pancreatic cancer based on REG4 or a combination of CA19-9 and REG4 also has stomach cancer or colon cancer can be easily ruled out.

The diagnosis and detection of early-staged pancreatic cancers has been very difficult, while the diagnosis or screening for other gastrointestinal (GI) malignancies y has been established by endoscopic or other non-invasive methods like fecal occult blood and serum pepsinogen which are well known in the art. If the claimed methods are applied to screen GI diseases and detect high level of serum REG4, endoscopic procedures can used to detect GI diseases, which are already established as reliable and sensitive methods. If no significant pathogenic lesion in the stomach or colorectum is detected by endoscopic study, invasive or non-invasive diagnostic procedures (Endoscopic Retrograde Cholangiopancreatography (ERCP), Endoscopic ultrasoundscopy (EUS), Magnetic resonance cholangiopancreatography (MRCP), etc), can then be used to detect early-staged pancreatic cancer. The prior art, however, does not provide any reliable tool to screen early-staged pancreatic cancer. For screening for other GI malignancies, fecal occult blood and serum pepsinogen are typically used. The presently claimed methods are a useful tool to screen pancreatic cancer by combining with other serum makers, e.g. CA19-9, and invasive endoscopic procedures.

Components used to carry out the diagnosis of pancreatic cancer according to the present invention can be combined in advance and supplied as a testing kit. Accordingly, the present invention provides a kit for detecting a pancreatic cancer, comprising:

(i) an immunoassay reagent for determining a level of REG4 in a blood sample; and

(ii) a positive control sample for REG4.

In the preferable embodiments, the kit of the present invention may further comprise:

(iii) an immunoassay reagent for determining a level of CA19-9 in a blood sample; and

(iv) a positive control sample for CA19-9.

The reagents for the immunoassays which constitute a kit of the present invention may comprise reagents necessary for the various immunoassays described above. Specifically, the reagents for the immunoassays comprise an antibody that recognizes the substance to be measured. The antibody can be modified depending on the assay format of the immunoassay. ELISA can be used as a preferable assay format of the present invention. In ELISA, for example, a first antibody immobilized onto a solid phase and a second antibody having a label are generally used.

Therefore, the immunoassay reagents for ELISA can comprise a first antibody immobilized onto a solid phase carrier. Fine particles or the inner walls of a reaction container can be used as the solid phase carrier. Magnetic particles can be used as the fine particles. Alternatively, multi-well plates such as 96-well microplates are often used as the reaction containers. Containers for processing a large number of samples, which are equipped with wells having a smaller volume than in 96-well microplates at a high density, are also known. In the present invention, the inner walls of these reaction containers can be used as the solid phase carriers.

The immunoassay reagents for ELISA may further comprise a second antibody having a label. The second antibody for ELISA may be an antibody onto which an enzyme is directly or indirectly linked. Methods for chemically linking an enzyme to an antibody are known. For example, immunoglobulins can be enzymatically cleaved to obtain fragments comprising the variable regions. By reducing the —SS— bonds comprised in these fragments to —SH groups, bifunctional linkers can be attached. By linking an enzyme to the bifunctional linkers in advance, enzymes can be linked to the antibody fragments.

Alternatively, to indirectly link an enzyme, for example, the avidin-biotin binding can be used. That is, an enzyme can be indirectly linked to an antibody by contacting a biotinylated antibody with an enzyme to which avidin has been attached. In addition, an enzyme can be indirectly linked to a second antibody using a third antibody which is an enzyme-labeled antibody recognizing the second antibody. For example, enzymes such as those exemplified above can be used as the enzymes to label the antibodies.

Kits of the present invention comprise a positive control for REG4. A positive control for REG4 comprises REG4 whose concentration has been determined in advance. Preferable concentrations are, for example, a concentration set as the standard value in a testing method of the present invention. Alternatively, a positive control having a higher concentration can also be combined. The positive control for REG4 in the present invention can additionally comprise CA19-9 whose concentration has been determined in advance. A positive control comprising both REG4 and CA19-9 is preferable as the positive control of the present invention.

Therefore, the present invention provides a positive control for detecting pancreatic cancer, which comprises both REG4 and CA19-9 at concentrations above a normal value. Alternatively, the present invention relates to the use of a blood sample comprising REG4 and CA19-9 at concentrations above a normal value in the production of a positive control for the detection of pancreatic cancer. It has been known that CA19-9 can serve as an index for pancreatic cancer; however, that REG4 can serve as an index for pancreatic cancer is a novel finding obtained by the present invention. Therefore, positive controls comprising REG4 in addition to CA19-9 are novel. The positive controls of the present invention can be prepared by adding CA19-9 and REG4 at concentrations above a standard value to blood samples. For example, sera comprising CA19-9 and REG4 at concentrations above a standard value are preferable as the positive controls of the present invention.

The positive controls in the present invention are preferably in a liquid form. In the present invention, blood samples are used as samples. Therefore, samples used as controls also need to be in a liquid form. Alternatively, by dissolving a dried positive control with a predefined amount of liquid at the time of use, a control that gives the tested concentration can be prepared. By packaging, together with a dried positive control, an amount of liquid necessary to dissolve it, the user can obtain the necessary positive control by just mixing them. REG4 used as the positive control can be a naturally-derived protein or it may be a recombinant protein. Similarly, for CA19-9 which is a carbohydrate antigen, a naturally-derived carbohydrate antigen or a chemically synthesized sialyl Lewis-type carbohydrate antigen can be used as the control. Not only positive controls, but also negative controls can be combined in the kits of the present invention. The positive controls or negative controls are used to verify that the results indicated by the immunoassays are correct.

The following examples are presented to illustrate the present invention and to assist one of ordinary skilled in the art in making and using the same. The examples are not intended in any way to otherwise limit the scope of the present invention.

Unless otherwise defined, all technical and scientific terms used herein have the same meaning as commonly understood by one of ordinary skilled in the art to which this invention belongs. Although methods and materials similar or equivalent to those described herein can be used in the practice or testing of the present invention, suitable methods and materials are described below. Any patents, patent applications, and publications cited herein are incorporated by reference.

Hereinbelow, the present invention will be specifically described using Examples, but it is not to be construed as being limited thereto.

### EXAMPLES

### Example 1

### Clinical Samples

Pre-operative and post-operative (one month after the operation) serum samples were obtained from seven patients undergoing pancreaticoduodenectomy for pancreatic adenocarcinoma under the appropriate rules for informed consent. Conventional paraffin-embedded tissue sections from PDACs were obtained from surgical specimens under the appropriate rules for informed consent. Serum samples were obtained from 59 pancreatic cancer patients, 35 other pancreatic diseases patients, and 56 normal healthy donors.

### Example 2

### Semi-Quantitative RT-PCR Analysis for REG4

Purification of PDAC cells and normal pancreatic ductal epithelium were described previously (Nakamura T, et al., (2004) Oncogene.; 23(13):2385-400); RNAs from the purified cell populations were subjected to two rounds of amplification by T7-based in vitro transcription (Epicentre Technologies, Madison, Wis.). The present inventors prepared appropriate dilutions of each single-stranded cDNA for subsequent PCR amplification by monitoring β-actin (ACTB) as quantitative control. The primer sequences were

| | |
|---|---|
| 5′-CATCCACGAAACTACCTTCAACT-3′ | (SEQ ID NO: 1) |
| and | |
| 5′-TCTCCTTAGAGAGAAGTGGGGTG-3′ | (SEQ ID NO: 2) |
| for ACTB; | |
| 5′-CCAATTGCTATGGTTACTTCAGG-3′ | (SEQ ID NO: 3) |
| and | |
| 5′-GAAAAACAAGCAGGAGTTGAGTG -3′ | (SEQ ID NO: 4) |
| for REG4. | |

All reactions were carried out under conditions of: initial denaturation at 94° C. for 2 min; and 21 cycles (for ACTB) or 28 cycles (for REG4) of 94° C. for 30 sec, 58° C. for 30 sec, and 72° C. for 1 min, on a GeneAmp PCR system 9700 (PE Applied Biosystems, Foster, Calif.).

### Example 3

### Production of Recombinant hREG4 (rhREG4)

### (1) Construction of Expression Cassette Vector

A target gene expression vector which coexpresses a target gene and a puromycin-EGFP fusion protein by IRES under the control of a CMV promoter was constructed.

KOD-Plus-(TOYOBO; KOD-201) was used for all PCR processes for gene amplification.

First, myc-His Tag gene was amplified from pcDNA3.1/myc-His A (Invitrogen; V800-2) by PCR using 5′-TTAATTAACTCGAGGGATCCCCCTTCGAACAAAAACTCATC-3′ (SEQ ID NO: 5) and 5′-GGCGAGAAAGGAAGGGAAG-3′ (SEQ ID NO: 6), and inserted into the SmaI site in pBluescriptII SK+ (TOYOBO) to construct pBlue/myc-His. pBlue/myc-His/EGFP was then prepared by inserting an EGFP gene amplified using 5′-ATCAGATCTATGGTGAGCAAGGGCGAGGA-3′ (SEQ ID NO: 7) and 5′-ATCTTACTTGTACAGCTCGTCCATGC-3′ (SEQ ID NO: 8) into the EcoRV site in pBlue/myc-His. Additionally, IRES-Puromycin gene sequence was amplified from pQCXIP (Clontech; 9136-1) using 5′-AATAGATATCCGCCCCTCTCCCTCCCC-3′ (SEQ ID NO: 9) and 5′-AATAGGATCCGGCACCGGGCTTGCG-3′ (SEQ ID NO: 10), and then digested with EcoRV and BamHI, and introduced into the PmeI-BglII site of pBlue/myc-His/EGFP to construct pBlue/myc-His/IRES-Puro-EGFP. Finally, the PacI-EcoRV fragment of pQCXIP was substituted with a gene segment of myc-His/IRES-Puro-EGFP excised from pBlue/myc-His/IRES-Puro-EGFP by PacI and EcoRV to construct the target gene expression vector pQCXmHIPG

### (2) Construction of REG4 mH Expression Vector

The REG4 gene was amplified by PCR using 5′-AATATTAATTAAGGAAGATGGCTTCCAGAAGCA-3′ (SEQ ID NO: 11) and 5′-AATAGGATCCTGGTCGGTACTTGCACAGGA-3′ (SEQ ID NO: 12), and then inserted into the PacI-BamHI site of pQCXmHIPG to construct pQC/REG4 mH/IPG.

### (3) Establishment of Expression Cell Line

Pantropic Retroviral Expression System (Clontech; K1063-1) was employed to establish an antigen-expressing cell line.

Confluent GP2-293 cells (Clontech; K1063-1) were prepared on collagen-coated 100 mm dishes, and cotransfected with 11.2 μg each of pQC/REG4 mH/IPG and pVSV-G (Clontech; K1063-1) using Lipofectamine 2000. After 48 hours, the supernatant comprising virus particles was collected, the viruses were precipitated by ultracentrifugation (18,000 rpm, 1.5 h, 4° C.), and the precipitate was suspended in 30 μL of TNE (50 mM Tris-HCl [pH 7.8], 130 mM NaCl, 1 mM EDTA) to prepare a retroviral vector solution. 5 μL of the retroviral vector solution was diluted with 150 μL of DMEM (SIGMA; D5796)-10% FBS containing 8 μg/mL Hexadimethrine bromide (SIGMA; H-9268), to prepare a medium containing virus particles. A medium in which 293T cells were grown on a to a confluency of about 40% in a 96-well plate was replaced with the prepared virus particle-containing medium, thereby introducing pQC/REG4 mH/IPG into the cells. After the gene introduction, the cells were cultured in DMEM (SIGMA; D5796)-10% FBS containing 5 μg/mL Puromycin (SIGMA; P-8833) to establish an expression cell line (REG4 mH/293T).

### (4) Purification of Antigen

About 1 L of culture supernatant of the established expression cell line was collected and used to purify His-tagged protein using TALON Purification Kit (Clontech; K1253-1). The purified protein was then dialyzed with PBS and confirmed by SDS-PAGE and Western blotting. The protein concentration was determined using Protein Assay Kit II (BioRad; 500-0002JA). This protein was taken to be the purified antigen.

### Example 4

### Polyclonal Antibody

rhREG4 protein was prepared for injection by emulsifying the antigen solution with adjuvant (Freund's complete adjuvant). Polyclonal anti-REG4 antibody (anti-REG4 pAb) was raised in rabbits (Medical & Biological Laboratories, Nagoya, Japan) against the purified full length of rhREG4 protein.

Affinity purification of the antisera was carried out as follows. Sepharose 4B (Amersham) resin was activated by bromocyan solution and coupled with rhREG4 protein. The filtered antiserum was added to the above-described resin, then washed with phosphate buffer (pH8.0) for 3 times. rhREG4-specific antibody was eluted by glycin-HCl buffer (pH2.3), neutralized with tris-HCl (pH8.0), and dialyzed with PBS.

### Example 5

### Monoclonal Antibody

### (1) Immunization

BALB/C mice (4 weeks old, female) (Japan SLC) were used as animals to be immunized, and the immunization was done by the foot pad method. 50 μL of an emulsion prepared by mixing 100 μL of immunogen adjusted to 0.1 mg/ml and an adjuvant (complete adjuvant (FREUND) Mitsubishi Kagaku latron RM606-1) was injected to both foot pads of four mice, respectively. The second and third (final) immunizations were carried out every three days (two-day intervals), and cell fusion was conducted three days after the third immunization.

### (2) Cell Fusion

Enlarged lymph nodes were excised from both feet of two of the immunized mice, the lymph nodes were cut, cells were pushed out by forceps or the like, and the cells obtained from the lymph nodes were collected by centrifugation. Myeloma cells (P3U1) were then mixed in at a rate of 2:1 to 10:1. The mixture was centrifuged and then 50% PEG (PEG4000; MERCK Cat No 1097270100) diluted with an equal volume of RPMI (RPMI1640; SIGMA Cat No R8758) was added to the obtained pellet to conduct the cell fusion. After washing, the cells were suspended in 160 mL of 15% FBS-HAT medium supplemented with HAT supplement (×50) (GIBCO Cat No 21060-017) and plated into sixteen 96-well plates at 200 μL/well. The medium was exchanged after three days, and after colony formation was confirmed (one to two weeks later), the first screening was carried out by immunoprecipitation.

### (3) Immunoprecipitation

50 μL of Protein G Sepharose beads washed in PBS were prepared in each well of a deep well plate, and 350 μL of the hybridoma culture supernatant was poured onto the beads to allow reaction for 1 hour at 4° C., under rotation. After washing with PBS, 350 μL of culture supernatant (nonspecifically bound substances adsorbed by Protein G Sepharose beads) was added as antigen to each well, and an antigen-antibody reaction was conducted under rotation for 1 hour at 4° C. The plate was washed again with PBS. 30 μL of 2× Sample Buffer was added to each well and boiled to prepare samples, and clones which can be immunoprecipitated were selected by detecting the tagged antigen by Western blotting.

### (4) Preparation of Monoclonal Hybridomas

The selected hybridomas were cloned by the limiting dilution method to obtain monoclonal hybridomas. The hybridomas were plated into a 96-well plate, culture supernatants of wells with single colonies were collected, and antibody activity was confirmed by the above immunoprecipitation. Cells in wells in which activity was confirmed were proliferated and clones 21-1, 24-1, and 34-1, which were strongly positive in immunoprecipitation, were obtained.

### (5) Antibody Purification

The culture supernatants of the hybridomas were applied onto Protein A columns at a rate of one drop per second to adsorb the antibodies, and washed with PBS/0.1% NaN₃ (until A280 became 0.05 or less when measured with an absorption spectrometer), and the adsorbed antibodies eluted by 0.5 M Arginine-HCl buffer (pH 4.1). In the elution step, ⅕ volume of 1 M Tris-HCl buffer (pH 8.0) was immediately added to the eluted samples for neutralization. After measuring A280 for each fraction, fractions of which A280 was 0.1 or more were pooled and dialyzed in PBS. After dialysis, the concentrations were determined based on the following formula: concentration=A280×0.7 [mg/mL].

### Example 6

### Immunohistochemical Staining

Tissue-microarray sections of pancreatic carcinomas (AccuMax Array) were purchased from Petagene Inc. (Seoul, Korea), where 31 PDAC tissues and 2 endocrine-tumor tissues were spotted in duplicate. The sections were deparaffinized and autoclaved for 15 min at 108° C. in citrate buffer, pH6.0. Endogenous peroxidase activity was quenched by incubation for 30 min in 0.33% hydrogen peroxide diluted in methanol. After incubation with fetal bovine serum for blocking, the sections were incubated with anti-REG4 polyclonal antibody for 1 h at room temperature. After washing with PBS, immunodetection was performed with peroxidase-labeled anti-mouse immunoglobulin (Envision kit, Dako Cytomation, Carpinteria, Calif.). Finally, the reactants were developed with 3,3′-diaminobenzidine (Dako) and the cells were counter-stained with hematoxylin.

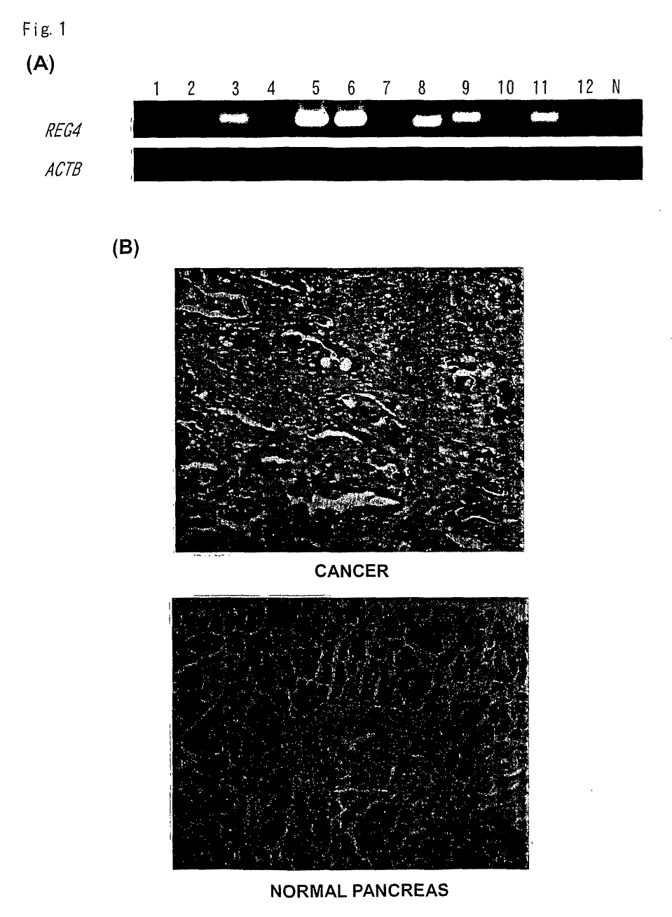

Immunohistochemical analysis using polyclonal antibody to REG4 at another series of PDAC tissues revealed strong signals of REG4 at the cytoplasm of cancer cells, while acinar cells in pancreas showed weak staining of REG4 (FIG. 1B). In addition, tissue-microarray with other series of 31 PDAC tissues spotted showed that 15 of 31 PDACs expressed high levels of REG4 (data not shown). Totally 35 out of 64 PDACs (55%) showed positive staining by anti-REG4 antibody (data not shown). This result is consistent with that from the previous RNA study of microdissected cells.

### Example 7

### ELISA Assay System

### (1) Sandwich ELISA System for Antibody Evaluation

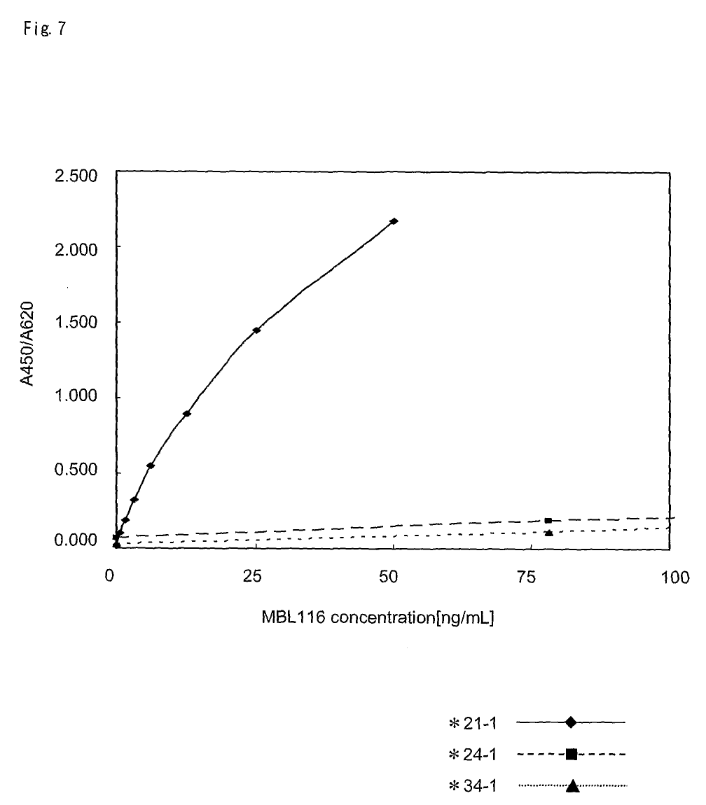

C8 MAXI NUNC-Immuno BreakApart Module (NUNC) was used as a microplate for the sandwich ELISA. Anti-REG4 monoclonal antibodies (clone 21-1, 24-1, and 34-1) were diluted with 0.1 M carbonate buffer (pH 9.6) to 10 μg/mL, added to the microplate at 50 μL/well, and left to stand overnight at 4° C. to immobilize each antibody by physical adsorption. After blocking with 1% BAS/PBS (RT, 2 hours), the blocking solution was discarded, and the residues were air-dried to prepare an assay plate. Specimens were diluted 5 times in reaction buffer (PBS, 0.1% Tween20, 1% BSA, pH 7.3), added to the assay plate at 50 μL/well, and reacted at room temperature for one hour. After washing four times with washing buffer (0.13% Tween20, 0.15 M NaCl/10 mM NaH₂PO₄), an HRP-labeled anti-REG4 polyclonal antibody was adjusted to 1.5 μg/mL with an enzyme-labeled antibody diluent (1% BAS, 0.135 M NaCl/20 mM HEPES) and added at 50 L/well. After reaction at the room temperature for one hour, the plate was washed four times with washing buffer. An enzyme substrate solution (Moss Inc.; TMB Ultra Sensitive Substrate) was added at 50 μL/well for coloring, and after 30 minutes, 0.36NH₂SO₄ was added at 50 μL/well to terminate the color reaction. The absorbance (A450/A620) was measured to calculate REG4 concentration in the serum based on the calibration curves of each antibody (FIG. 7).

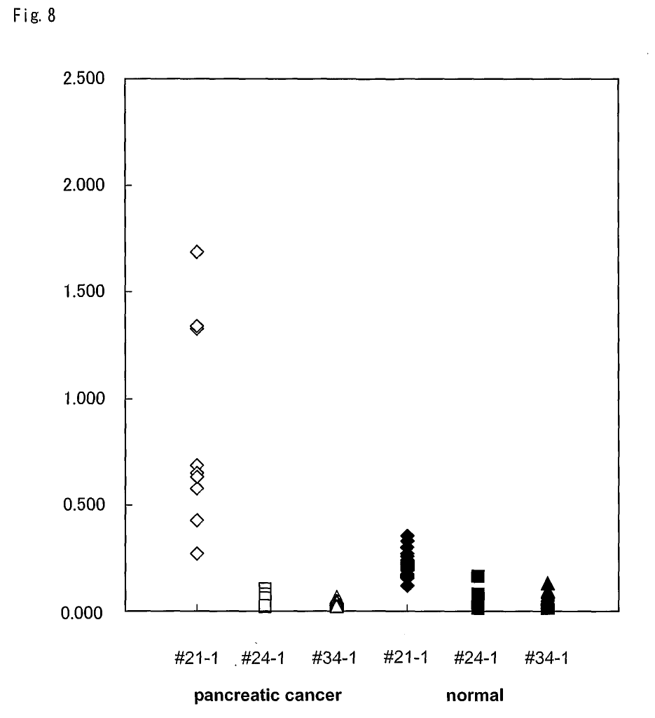

Using the above sandwich ELISA system, REG4 concentration was determined in specimens from 9 patients with pancreatic cancer and 28 healthy subjects to evaluate the antibody titer of each clone. The detection sensitivity of clones 24-1 and 34-1 was low as compared to clone 21-1 (FIG. 7), and REG4 in the specimens of pancreatic cancer patients could be detected only in clone 21-1 (FIG. 8). In addition, REG4 concentration in specimens measured by the sandwich ELISA system using clone 21-1 showed a high value in pancreatic cancer patients as compared to healthy subjects, confirming a significant difference (P<0.05) in REG4 concentrations in specimens from healthy subjects and pancreatic cancer patients (FIG. 8).

Amino acid sequences of the variable regions and each CDR in clone 21-1, and nucleotide sequences of DNAs encoding them are as follows:

| | Nucleotide sequence | Amino acid sequence |
|---|---|---|
| Heavy Chain | SEQ ID NO: 15 | SEQ ID NO: 16 |
| CDR1 | SEQ ID NO: 17 | SEQ ID NO: 18 |
| CDR2 | SEQ ID NO: 19 | SEQ ID NO: 20 |
| CDR3 | SEQ ID NO: 21 | SEQ ID NO: 22 |
| Light Chain | SEQ ID NO: 23 | SEQ ID NO: 24 |
| CDR1 | SEQ ID NO: 25 | SEQ ID NO: 26 |
| CDR2 | SEQ ID NO: 27 | SEQ ID NO: 28 |
| CDR3 | SEQ ID NO: 29 | SEQ ID NO: 30 |

Further, the heavy chain and light chain were found to have signal sequences as shown in SEQ ID NOs: 32 and 34, respectively. Nucleotide sequences of cDNAs encoding the variable regions of the heavy chain and light chain, which comprise signal sequences, are shown in SEQ ID NOs: 31 and 33.

### (2) Sandwich ELISA System

As a microplate for the sandwich ELISA system, C8 MAXI NUNC-Immuno BreakApart Module (NUNC) was used. An anti-REG4 monoclonal antibody (clone 21-1) was diluted with 0.1 M carbonate buffer (pH 9.6) to 10 μg/mL, added to the microplate at 100 μL/well, and left to stand overnight at 4° C. to sensitize the antibody by physical adsorption. After blocking (RT, 2 hours), the blocking solution was discarded, and the residues were dried to prepare an assay plate. The sera from patients were diluted 5 times in a specimen diluent (PBS, 0.1% Tween20, 1% BSA, pH 7.3) to which 0.5 μg/mL of biotinylated anti-PEG4 polyclonal antibody had been added. After reaction for 15 minutes, the sera were added to the assay plate at 100 μL/well and reacted for 2 hours. After washing five times, 8000 times diluted HRP-labeled streptavidin (Amersham; RPN4401) was added at 100 μL/well, reacted for one hour, and then washed five times. 100 μL/well of TMB substrate solution (MOSS Inc.; TMB Ultra Sensitive Substrate) was added for coloring. After 15 minutes, 100 μL/well of 0.18 M sulfuric acid was added to terminate the color reaction, and REG4 concentration in the preoperative and postoperative sera obtained from the seven patients was determined using the absorbance (A450/A620).

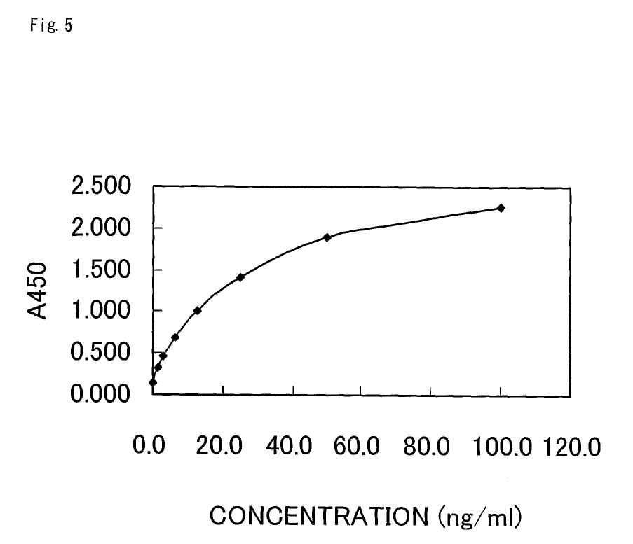

The above sandwich ELISA system was further improved, and the REG4 concentration in the sera of 59 patients with pancreatic cancer, 35 patients with inflammatory pancreatic disease, and 56 healthy subjects, was determined by the following sandwich ELISA system. C8_MAXI NUNC-Immuno BreakApart Module (NUNC) was used as a microplate. An anti-REG4 monoclonal antibody (clone 21-1) was diluted with 0.1 M carbonate buffer (pH 9.6) to 10 μg/mL, added to the microplate at 50 μL/well, and left to stand overnight at 4° C. to sensitize the antibody by physical adsorption. After blocking with 1% BSA/PBS (RT, 2 hours), the blocking solution was discarded, and the residues were dried to prepare an assay plate. The sera from the patients were diluted five times with a specimen diluent (PBS, 0.1% Tween20, 1% BSA, pH 7.3) to which 0.5 μg/mL of biotinylated anti-REG4 polyclonal antibody had been added. After reaction for 15 minutes, the sera were added to the assay plate at 50 μL/well and reacted for 1 hour. After washing four times with washing buffer (0.13% Tween20, 0.15 M NaCl/10 mM NaH2PO4), an REG4-specific polyclonal antibody was adjusted with the reaction buffer (same as the above) to 0.25 μg/mL, added at 50 μL/well, and reacted for one hour at room temperature. After washing four times with the washing buffer, HRP-labeled streptavidin (Amersham; RPN4401) diluted 150,000 times with an enzyme-labeled antibody diluent (1% BSA, 0.135 M NaCl/20 mM HEPES) was added at 50 μL/well, reacted at room temperature for one hour, and then washed four times with the washing buffer. 50 μL/well of TMB substrate solution (Moss Inc.; TMB Ultra Sensitive Substrate) was added and this was left to stand at room temperature for 30 minutes for coloring. 50 μL/well of 0.36N sulfuric acid was added to terminate the color reaction, and then the absorbance (A450/620) was measured to determine REG4 concentration in the sera using a calibration curve (FIG. 5).

### (3) Serum REG4 Level Measured by ELISA

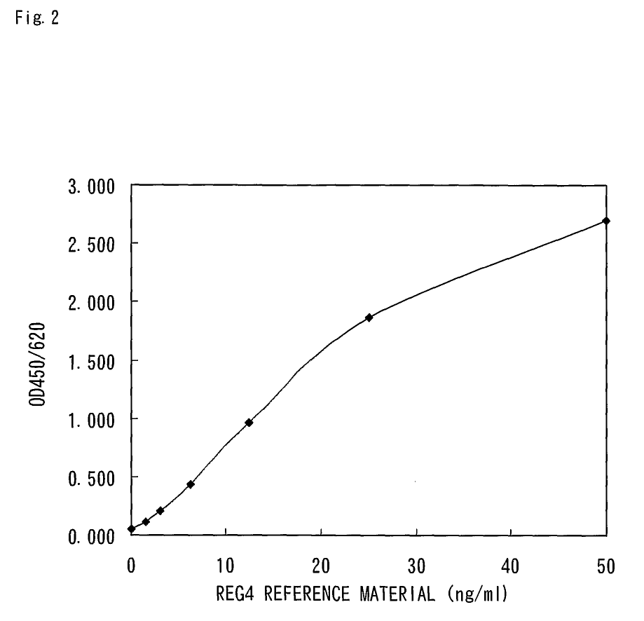

REG4 is a secreted protein. The present inventors validated that REG4 protein was secreted into the culture medium of pancreatic cancer cell lines by immunoprecipitation using antibodies generated by the present inventors (data not shown). In order to measure REG4 level in serum of pancreatic cancer patients, the present inventors established sandwich ELISA system using mouse monoclonal antibody (clone 21-1), which reveled the strongest affinity to human REG4, and rabbit polyclonal antibody to human REG4. The performance characteristics of the sandwich ELISA (standard curve) was shown in FIG. 2. In addition, the present inventors established the modified sandwich ELISA using these antibodies. The performance characteristics of the modified sandwich ELISA (standard curve) was shown in FIG. 5.

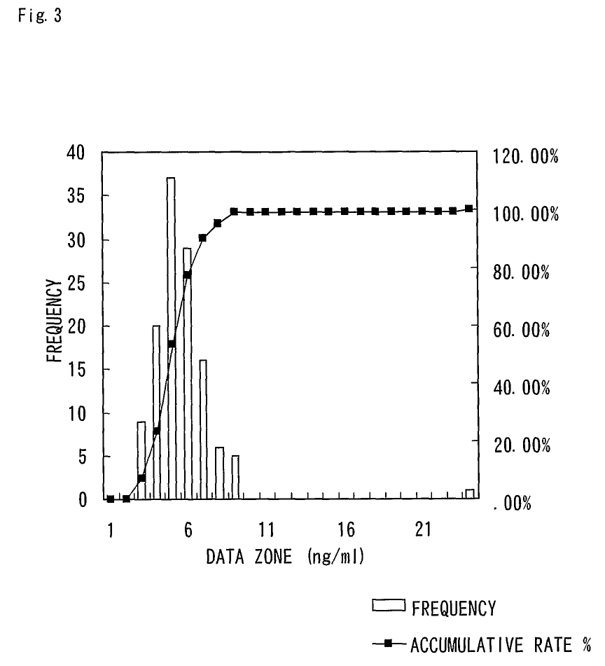

To determine the sensitivity of elevated REG4 as a diagnostic test, the present inventors measured serum REG4 of 123 healthy people and defined a cutoff value of 9.0 ng/ml, a level 2 SDs above the mean REG4 level in these healthy controls. (FIG. 3)

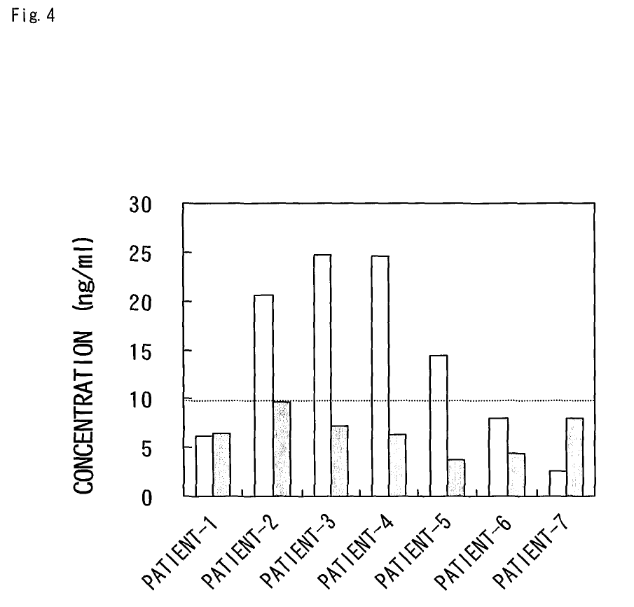

Then the present inventors analyzed pre-operative and post-operative serum from seven patients with operable pancreatic adenocarcinoma (FIG. 4). Four out of these seven cases showed more than 9.0 ng/ml REG4 level at pre-operation (Case 2, 3, 4, and 5) and REG4 levels of these four cases fell down to the normal range of REG4 level four weeks after the resection of their tumors. These results suggest that serum REG4 was derived from pancreatic cancer tissues and REG4 was potentially a serum marker for pancreatic cancers. The rest cases showed less than 9.0 ng/ml of REG4 both at pre-operation and post-operation.

Furthermore, the present inventors measured serum REG4 of 59 pancreatic cancer cases, 35 other pancreatic disease cases, and 56 normal healthy people using modified sandwich ELISA. There was the significant difference between the pancreatic cancer cases and normal healthy people (p<0.01), and between the pancreatic cancer cases and the other pancreatic disease (p<0.05) cases. To determine the sensitivity of elevated REG4 as a diagnostic test, the present inventors defined a cutoff value 3.78 ng/ml, a level 3 SDs above the mean REG4 level in these healthy controls. As a result, 29 of 59 pancreatic cancer cases (49.2%), 10 of 35 other pancreatic disease cases (28.6%), and 1 of 56 normal healthy controls (1.8%) were judged as positive. On the other hand, 45 of 59 pancreatic cancer cases (76.3%), 13 of 35 other pancreatic disease cases (37.1%), and 5 of 56 normal healthy controls (8.9%) were judged as positive by the ELISA system for detecting CA19-9 (cutoff value 25 ng/ml) (CA19-9 EIA Kit; CanAg Diagnostics AB). At least one of the two proteins was positive in 52 of 59 pancreatic cancer cases (88.1%), 19 of 35 other pancreatic disease cases (54.3%), and 6 of 56 normal healthy cases (10.7%) (FIG. 6).

Herein, the present inventors found that approximately a half of PDACs showed overexpression of REG4 protein and that serum REG4 could be detected in some patients with PDACs by ELISA system constructed by the present inventors. In Table 2, the present inventors summarized the clinicopathological features and pre-operative serum levels of REG4, CA19-9, and CEA in the seven cases examined by the present inventors, and four out of these seven cases showed higher level of serum REG4 than normal healthy control (more than 9.0 ng/ml). Interestingly, serum REG4 was at high level in the patients with early-staged or non-invasive pancreatic cancer (Case 3 and 4) and also in the patients who survived longer (Case 3, 4, and 5), suggesting that there might be much potential that serum REG4 could detect in the PDAC patients who would be expected to have early-staged cancer or good prognosis. Serum CA19-9 and CEA did not find these early-staged or non-invasive cases and serum REG4 can be a promising serum marker to screen pancreatic cancer.

TABLE 2
Serum marker levels and clinicopathological characteristics

| | | | | | | | CA | | |
|---|---|---|---|---|---|---|---|---|---|
| Case | Age | Location | TNM | Stage | Histology | REG4¹⁾ | 19-²⁾ | CEA³⁾ | Prognosis⁴⁾ |
| 1 | 56 | Head | T2N1M0 | III | Poorly differentiated | 6.2 | 84 | 1.3 | 14 M dead |
| | | | | | tubular adenoca | | | | |
| 2 | 64 | Head | T2N1M0 | III | Moderate differentiated | 20.5 | 1945 | 12.1 | 9 M dead |
| | | | | | tubular adenoca | | | | |
| 3 | 69 | Head | T2N0M0 | I | Intraductal tubular | 24.7 | 24 | 4.2 | 14 M alive |
| | | | | | adenoca | | | | |
| 4 | 78 | Head | T1N0M0 | I | Intraductal papillary | 24.6 | 16 | 2.8 | 18 M alive |
| | | | | | mucinous carcinoma | | | | |
| 5 | 56 | Head | T2N1M0 | III | Moderate differentiated | 14.5 | 311 | 1.6 | 13 M alive |
| | | | | | tubular adenoca | | | | |
| 6 | 68 | Tail | T2N0M0 | I | Moderate differentiated | 8.0 | 5 | 1.0 | 8 M dead |
| | | | | | tubular adenoca | | | | |
| 7 | 70 | Head | T2N1M0 | III | Poorly differentiated | 2.5 | 17 | 4.6 | 3 M dead |
| | | | | | tubular adenoca | | | | |

¹⁾Normal range <9.0 ng/ml
²⁾Normal range <36 U/ml
³⁾Normal range <5.0 ng/ml, value above the normal range of each marker is underlined
⁴⁾M: month

The sensitivity and specificity of serum REG4 as a tumor marker of PDACs should be determined by analyzing large-numbered studies. Some previous studies reported that REG4 expression in colorectal cancer (Violette S, et al., (2003) Int J. Cancer.; 103(2):185-93), gastric cancer (Oue N, et al., (2005) Cancer Res.; 64(7):2397-405) and inflammatory bowel diseases (Hartupee J C, et al., (2004) Biochim Biophys Acta.; 1518(3):287-93), and further studies are required to examine whether serum REG4 could distinguish pancreatic cancer from these diseases. In addition, chronic pancreatitis is one of the benign diseases to be distinguished from PDACs by the present inventors. Considering that REG family is likely to be associated with tissue regeneration, the present inventors also need analyzing the serum from patients with chronic pancreatitis. However, pancreatic cancers, especially early-staged PDACs, are extremely hard to detect, while other bowl or gastric lesions that may be associated with high level of serum REG4 are easily detected by endoscopic examination, and even if serum REG4 is elevated in these digestive diseases, serum REG4 measurement is thought to be valuable to screen pancreatic cancers. Like other tumor markers, serum REG4 may not have enough ability to detect all cases of PDACs or to distinguish PDACs from other diseases, but combining serum REG4 with other tumor markers such as CA19-9 or diagnostic imaging could provide us with promising ability to approach to detect early-staged or precursor lesions of PDACs and screen these diseases more efficiently.

### INDUSTRIAL APPLICABILITY

The present invention involves the discovery that REG4 levels are elevated in the sera of pancreatic-cancer patients as compared to normal controls. Accordingly, the REG4 protein has utility as a diagnostic marker (i.e. serum). Using the level of REG4 as an index, the present invention provides methods for diagnosing, and monitoring the progress of cancer treatment, of cancer patients. The prior art fails to provide a suitable serological marker for pancreatic cancer. Novel serological marker REG4 of the present invention may improve the sensitivity for detection of pancreatic cancer. In addition, the combination of REG4 and CA19-9 contributes to increase the sensitivity for detecting pancreatic cancer.

While the present invention has been described in detail and with reference to specific embodiments thereof, it will be apparent to one skilled in the art that various changes and modifications can be made therein without departing from the spirit and scope of the invention.

Unless otherwise defined, all technical and scientific terms used herein have the same meaning as commonly understood by one of ordinary skilled in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods and examples are illustrative only and not intended to be limiting.

The invention has been illustrated by reference to specific examples and preferred embodiments. It should be understood that the invention is intended not to be limited by the foregoing description, but to be defined by the appended claims and their equivalents.

1. A method for diagnosing pancreatic cancer in a subject, comprising the steps of:
(a) providing a blood sample from a subject to be diagnosed;
(b) determining a level of REG4 in the blood sample;
(c) comparing the REG4 level determined in step (b) with that of a normal control, wherein a high REG4 level in the blood sample, compared to the normal control, indicates that the subject suffers from pancreatic cancer.

2. The method of claim 1, wherein the blood sample is selected from the group consisting of whole blood, serum, and plasma.

3. The method of claim 1, wherein the REG4 level is determined by detecting the REG4 protein in the blood sample.

4. The method of claim. 3, wherein the REG4 protein is detected by immunoassay.

5. The method of claim 4, wherein the immunoassay is an ELISA.

6. The method of claim 4, wherein the immunoassay is sandwich method which uses an anti-REG4 monoclonal antibody immobilized on a carrier.

7. The method of claim 6, wherein the monoclonal antibody comprises a VH and VL chain, each VH and VL chain comprising CDR amino acid sequences designated CDR1, CDR2 and CDR3 separated by framework amino acid sequences, the amino acid sequence of each CDR in each VH and VL chain is selected from the group consisting of:
VH CDR1: SEQ ID NO: 18
VH CDR2: SEQ ID NO: 20
VH CDR3: SEQ ID NO: 22
VL CDR1: SEQ ID NO: 26
VL CDR2: SEQ ID NO: 28 and
VL CDR3: SEQ ID NO: 30,
or a fragment comprising antigen binding region thereof.

8. The method of claim 7, wherein the VH comprises the amino acid sequence of SEQ ID NO: 16, and VL comprises the amino acid sequence of SEQ ID NO: 24.

9. The method of claim 1, further comprising the steps of:
(d) determining a level of CA19-9 in the blood sample;
(e) comparing the CA19-9 level determined in step (d) with that of a normal control, wherein either or both of high REG4 and high CA19-9 levels in the blood sample, compared to the normal control, indicate that the subject suffers from pancreatic cancer.

10. An immunoassay reagent for detecting REG4 in a blood sample, wherein the reagent comprises an anti-REG4 antibody.

11. The reagent of claim 10, wherein the monoclonal antibody is immobilized on a carrier.

12. The reagent of claim 11, wherein the anti-REG4 antibody comprises a monoclonal antibody comprises a VH and VL chain, each VH and VL chain comprising CDR amino acid sequences designated CDR1, CDR2 and CDR3 separated by framework amino acid sequences, the amino acid sequence of each CDR in each VH and VL chain is selected from the group consisting of:
VH CDR1: SEQ ID NO: 18
VH CDR2: SEQ ID NO: 20
VH CDR3: SEQ ID NO: 22
VL CDR1: SEQ ID NO: 26
VL CDR2: SEQ ID NO: 28 and
VL CDR3: SEQ ID NO: 30,
or a fragment comprising antigen binding region thereof.

13. The reagent of claim 12, wherein the VH comprises the amino acid sequence of SEQ ID NO: 16, and VL comprises the amino acid sequence of SEQ ID NO: 24.

14. A kit for detecting a pancreatic cancer, wherein the kit comprises:
(i) an immunoassay reagent for determining a level of REG4 in a blood sample; and
(ii) a positive control sample for REG4.

15. The kit of claim 14, which further comprises:
(iii) an immunoassay reagent for determining a level of CA 19-9 in a blood sample; and
(iv) a positive control sample for CA19-9.

16. The kit of claim 15, wherein the positive control sample is positive for both of REG4 and CA19-9.

17. The kit of claim 16, wherein the positive control sample is liquid form.

18. A positive control blood sample for detecting a pancreatic cancer, wherein the blood sample comprises more than normal level of both of REG4 and CA19-9.

19. An anti-REG4 monoclonal antibody comprises a VH and VL chain, each VH and VL chain comprising CDR amino acid sequences designated CDR1, CDR2 and CDR3 separated by framework amino acid sequences, the amino acid sequence of each CDR in each VH and VL chain is selected from the group consisting of:
VH CDR1: SEQ ID NO: 18
VH CDR2: SEQ ID NO: 20
VH CDR3: SEQ ID NO: 22
VL CDR1: SEQ ID NO: 26
VL CDR2: SEQ ID NO: 28 and
VL CDR3: SEQ ID NO: 30,
or a fragment comprising antigen binding region thereof.

20. The monoclonal antibody of claim 19, wherein the VH comprises the amino acid sequence of SEQ ID NO: 16, and VL comprises the amino acid sequence of SEQ ID NO: 24.
